(19) United States
(12) Patent Application Publication
(10) Pub. No.: US 2026/0083918 A1
(43) Pub. Date: Mar. 26, 2026

(54) TAMPER RESISTANT AND TAMPER EVIDENT SYRINGE ASSEMBLY

(71) Applicant: CIVICA, INC., Lehi, UT (US)

(72) Inventors: Brian George, Lehi, UT (US); Alex Crawford, Lehi, UT (US)

(73) Assignee: CIVICA, INC., Lehi, UT (US)

(21) Appl. No.: 18/895,471

(22) Filed: Sep. 25, 2024

(51) Int. Cl. A61M 5/32; A61M 5/315

(52) U.S. Cl. CPC A61M 5/3202; A61M 5/31501

(57) ABSTRACT

Devices and methods are disclosed herein for a tamper evident and tamper resistant device for a syringe. The small size of the device reduces costs related to production as well as preserves space in medical carts or other medical storage areas. The device includes two ends connected via a shaft. The two ends are shaped to be complementary to each end of the syringe, particularly to cover the ends of a pre-filled syringe to prevent access for potential tampering. Also disclosed is a wrapper and method of wrapping the device and the syringe where removal of the wrapping or attempted access to the syringe provides evidence of tampering. The wrapping can also be perforated to allow for ease of removal during actual intended use of the pre-filled syringe.

### BACKGROUND

### Field

The present disclosure is directed to a tamper evident and tamper resistant assembly which is designed to restrict access to contents of a loaded or pre-filled syringe by preventing rotation or removal of the syringe plunger rod and restricting access to the plunger itself by preventing access to the interior of the syringe barrel through the open end. The assembly additionally prevents access to the tip or cap of the pre-filled syringe by physically blocking access to the tip or cap, with or without a luer-lock cap device.

The assembly additionally is enveloped in shrink wrap to indicate tampering. The shrink wrap may be perforated along the length of the device to allow for ease of removal. The shrink wrap, in connection with the entire tamper evident or tamper resistant assembly, serve to prevent tampering, and show evidence of the same.

### Description of Related Art

Administering injections in patients is a common activity in the medical field, yet presents a number of risks and challenges for users and healthcare professionals. The most conventional form of injectable drug delivery involves the combination of a vial and a hypodermic needle. However, more recent innovations have advanced administration of injectable drugs via prefilled syringes.

The use of prefilled syringes has increased the speed and accuracy of injectable drug delivery by allowing nurses or medical professionals a quick and easy way to inject the medicament. Prefilled syringes have four key components; a plunger rod, a plunger, a barrel, and a needle. For the mechanism to function properly, the plunger rod and stopper must fit tightly inside of the barrel, but must also glide through when pressure is applied. Prefilled syringes may be made of glass, plastic, rubber, and/or silicone materials.

As it is relatively common for medical personnel to prescribe an injectable drug into a patient, it is also common to have prefilled syringes in medical settings. Prefilled syringes may be used with medicaments which are very expensive or addictive, such as opiates, and there is a danger that the prefilled syringe or other device may be tampered with at some point in the administration process, between filling in a pharmacy to transportation to the subject in need. This possibility can present true dangers if a person seeking unauthorized access to the drug is able to then gain access to the device and tamper with it in some manner. For example, an unauthorized person could extract the desired medicament and replace with saline solution or water. Alternatively, they may tamper with the medicament and add other destructive or potentially harmful chemicals.

Therefore, there exists a need to prevent tampering with a prefilled device and to provide a method of proof of tampering should it occur. To overcome disadvantages and problems of using a prefilled syringe, a variety of tamper evident assemblies have been proposed. Many of these assemblies are structured to prevent or restrict access to the contents of a prefilled syringe, mostly by completely encapsulating or covering the syringe with another package. These assemblies intend to provide clear indication of having been tampered with because of the obvious evidence shown if they are broken or opened.

However, many of said tamper resistant or evident assemblies are not practical for use by medical professionals. Their large size and use of expensive materials takes up valuable space in medical carts, which also in turn increases costs.

Additionally, those who wish to tamper with the devices have discovered ways to tamper with the drugs which are harder to detect. Specifically, some may use an additional needle to insert into the rubber plunger by gaining access through the opening in which the plunger rod is placed. The availability to access the vulnerable plunger by outside means opens the door to tampering which may not at first be noticed.

As will become clear from the description below, the components of the tamper resistant and evident assembly operate individually or collectively to both provide clear evidence of tampering of the prefilled device, and to prevent access to the vulnerable portions of the device.

### SUMMARY

The present disclosure intends to present solutions to the above identified deficiencies and needs within the art, and is directed to a tamper resistant and tamper evident assembly to prevent access to the medicaments within a prefilled syringe. Specifically, access is physically blocked to the interior of the syringe and thus the plunger and plunger rod, to prevent rotation or removal of said plunger rod, to prevent access from the syringe tip or cap covering, and to provide a method of visually showing that the prefilled syringe has or has not been tampered with.

As will become clear from the description below, one or more components of the tamper resistant and tamper evident assembly operate individually or in connection with a prefilled syringe to provide clear evidence or indication of tampering, in the case that unauthorized access to the medicament has been attempted. Similarly, authorized use and dispelling of the medicaments within the syringe will be clearly evident.

The assembly as proposed is described as used nonexclusively, but adapted for use with a prefilled syringe which is commonly used.

### BRIEF DESCRIPTION OF THE DRAWINGS

Other features and advantages of the assemblies disclosed herein will be apparent to those skilled in the art reading the following detailed description in conjugation with the exemplary embodiments illustrated in the drawings, wherein:

FIG. 1 shows a perspective view of the tamper evident and/or tamper resistant assembly according to an embodiment of the present invention illustrated;

FIG. 2 shows a perspective view of the tamper evident and/or tamper resistant assembly as connected to and assembled on a prefilled syringe;

FIG. 3 shows a front view of the tamper evident and/or tamper resistant assembly;

FIG. 4 shows a front view of the tamper evident and/or tamper resistant assembly as connected to and assembled on a prefilled syringe;

FIG. 5 shows a back view of the tamper evident and/or tamper resistant assembly;

FIG. 6 shows a back view of the tamper evident and/or tamper resistant assembly; as connected to and assembled on a prefilled syringe;

FIG. 7 shows a side view of the tamper evident and/or tamper resistant assembly;

FIG. 8 shows a side view of the tamper evident and/or tamper resistant assembly as connected to and assembled on a prefilled syringe;

FIG. 9 shows a top view of the proximal end of the tamper evident and/or tamper resistant assembly;

FIG. 10 shows a top view of the tamper evident and/or tamper resistant assembly as connected to and assembled on a prefilled syringe;

FIG. 11 shows a bottom view of the distal end of the tamper evident and/or tamper resistant assembly;

FIG. 12 shows a bottom view of the tamper evident and/or tamper resistant assembly as connected to and assembled on a prefilled syringe; and,

FIG. 13 shows a perspective view of a tamper evident and/or tamper resistant assembly according to another embodiment of the invention.

### DESCRIPTION OF THE INVENTION

The following provides a description of various exemplary embodiments of the disclosure, as used in the context of a tamper evident and/or tamper resistant device as may or may not be connected to a prefilled syringe. It will be appreciated that these examples are not limiting.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The use of any and all examples, or exemplary language (e.g., “such as”) provided herein, is intended merely to better illuminate the invention, and does not pose a limitation on the scope of the invention unless otherwise claimed. No language in the specification should be construed as indicating any non-claimed element as essential to the practice of the invention.

The present disclosure is directed to a tamper evident assembly for preventing access to the contents of a pre-filled syringe, specifically by preventing access to the interior of a syringe, to protect the medicine or other contents therein. The tamper evident assembly is disposed and structured to restrict access to the opening tip, restrict removal of the plunger rod from the interior of the barrel of the syringe, and prevent access to the vulnerable rubber plunger.

As will become clear from the following description, at least one or more of the components of the tamper evident device operate either individually or in combination to provide a clear indication of tampering with said pre-filled syringe if an unauthorized attempt at access has occurred. Similarly, any authorized use of the syringe will be evident.

The tamper evident device is specifically, although not exclusively, adapted for use with a loaded or “pre-filled” syringe as commonly used in the medical profession. Prevention of access from either side of said syringe, including the piston or stopper, restricts access to the medicine or other contents within the syringe through any possible open end of the syringe, which may otherwise occur if the tamper evident device was not present.

As represented throughout the Figures, the present disclosure is directed to embodiments of a tamper resistant and/or tamper evident assembly 100 for a syringe, structured to prevent or restrict access to the interior components of a syringe generally depicted as 105 which is, but not limited to, a pre-filled syringe containing a drug, medicine, or other liquid content.

The pre-filled syringe 105 may additionally have finger flanges 116 through which an operator would hold the syringe 105 while dispensing.

The pre-filled syringe 105 may be any standard sized pre-filled syringe, such as a 1 mL short syringe, or may be of various sizes, such as 2.25 mL or 5 mL.

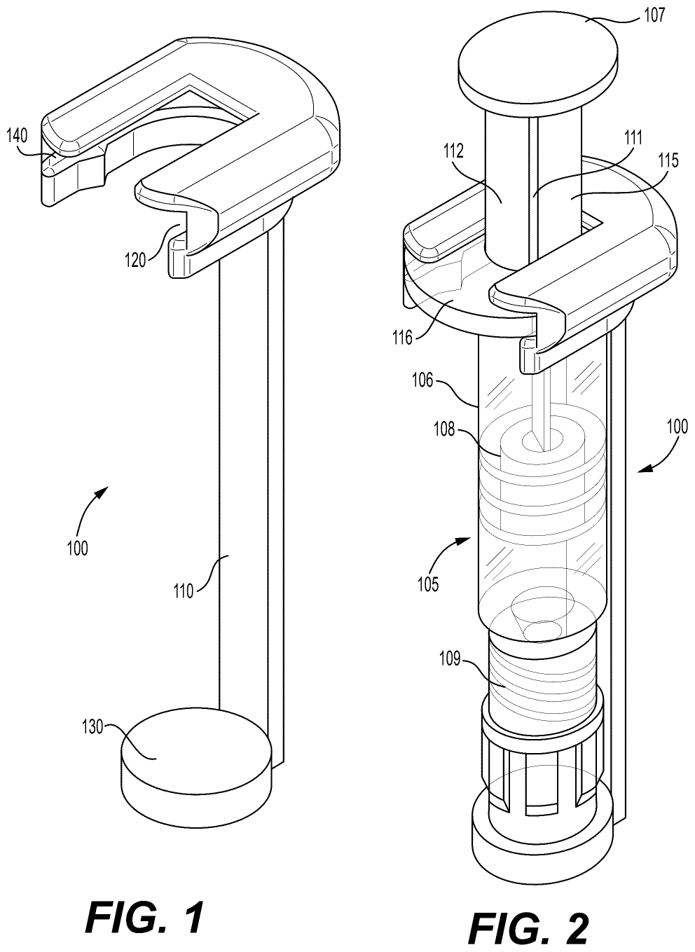

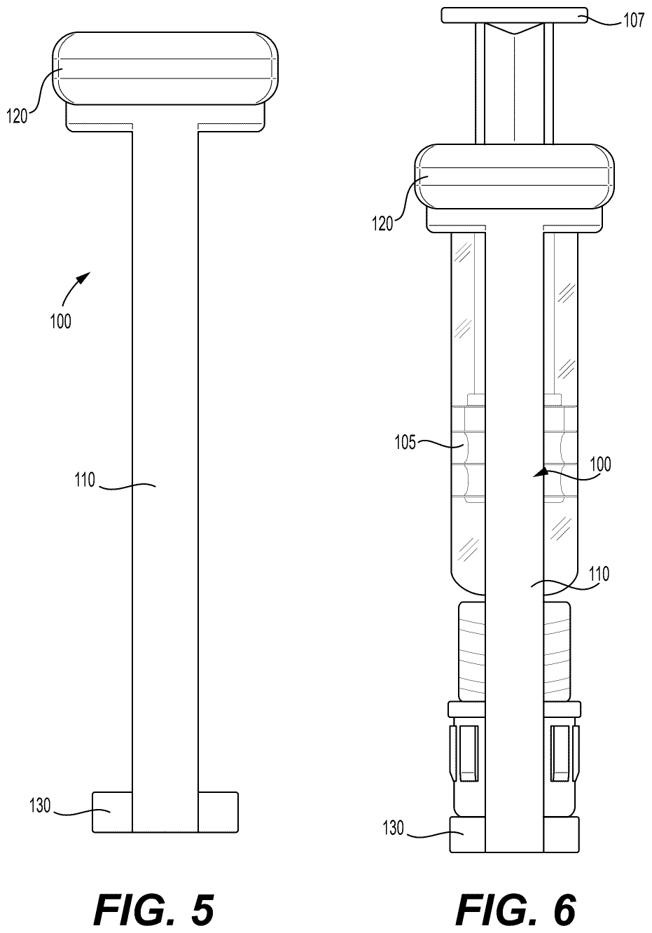

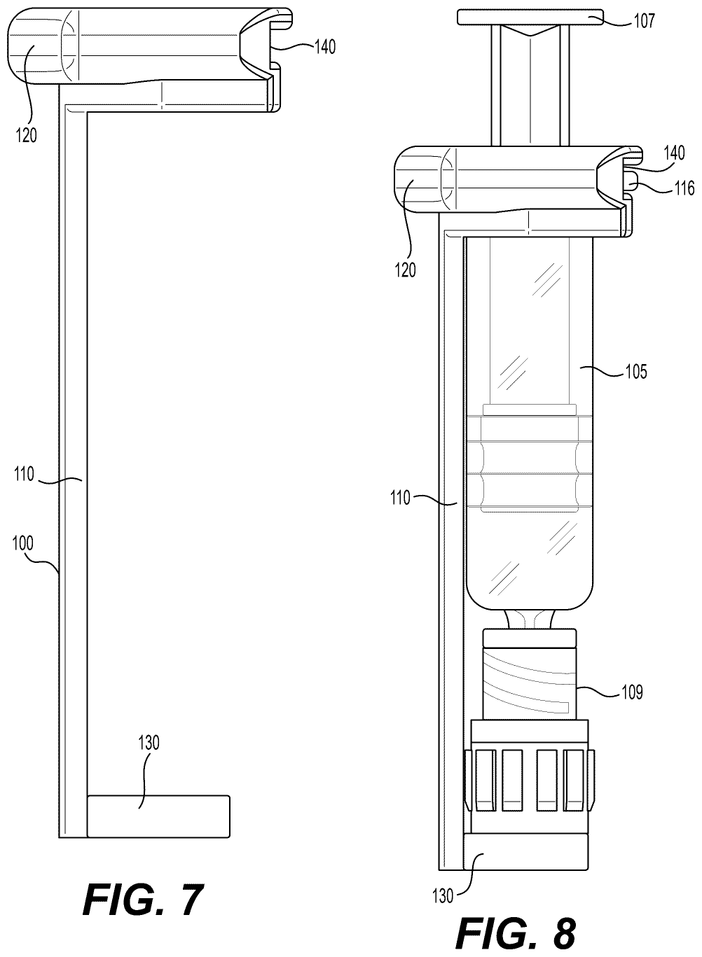

As may be seen in at least FIGS. 1, 3, 5, 7, and 9 the unassembled tamper resistant assembly 100 generally may have an elongated shaft 110 which connects a proximal end 120 and a distal end 130 of the assembly 100.

As shown in FIGS. 2, 4, 6, and 8, the assembly 100 may be disposed in cooperative connection, attachment, or other direct association with the pre-filled syringe 105. The pre-filled syringe 105 may comprise a syringe body 106, a plunger rod 107, and a tip cover 109 which may be, for example, in the form of a luer lock cover. The plunger rod 107 may comprise a plunger shaft 115 and a plunger 108.

The distal end 130 of the assembly 100 is structured to engage and enclose or cover the tip cover 109 of the syringe 105. The tip cover 109 may be referred to as a “fitting” of the syringe, which covers any nozzle or discharge port. The distal end 130 and the tip cover 109 are meant to be placed in close relation or adjacent to one another to prevent access to the tip cover 109 which may cover any nozzle or discharge port of the syringe 105.

The distal end 130 is connected non-removably at an end of the elongated shaft 110. The distal end 130 protrudes horizontally or perpendicularly from the elongated shaft 110 in a location that allows close connection to the tip cover 109. The distal end 130 additionally may extend a length far enough from the elongated shaft 110 such that the length of the distal end 130 horizontally allows for full coverage of the tip cover 109 or such length that a nozzle may be on the tip cover 109, such as a luer lock fitting.

The proximal end 120 of the assembly 100 is connected non-removably at an opposite end of the elongated shaft 110. The proximal end 120 protrudes horizontally or perpendicularly from the elongated shaft 110 in an outward direction that is complimentary to the direction of protrusion of the distal end 130. The proximal end 120 by virtue of its size may additionally protrude a distance opposite the protrusion of the distal end 130 to accommodate for the size of a finger flange 116 of the syringe 105.

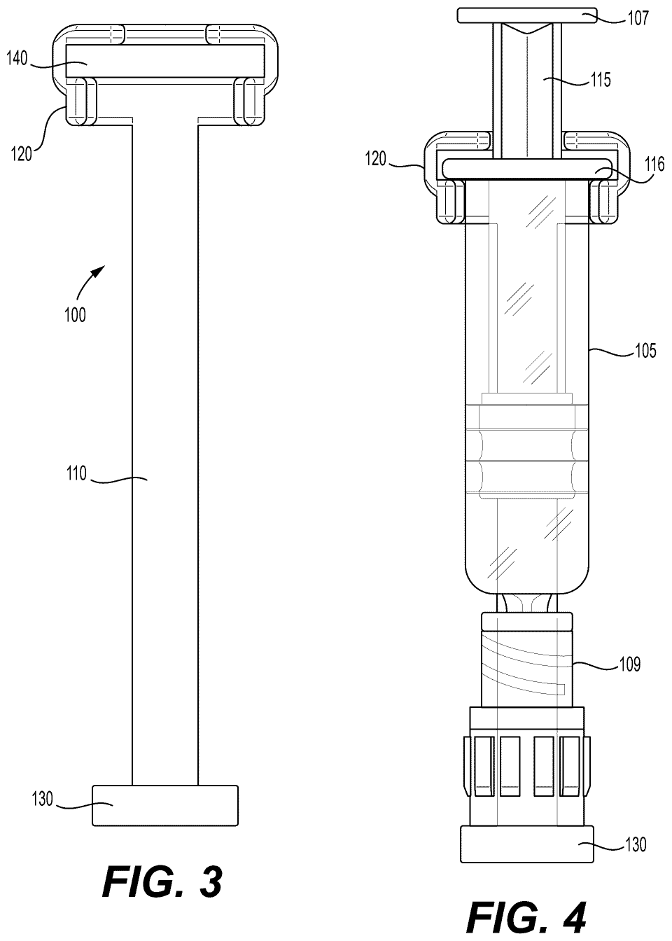

The proximal end 120 of the assembly 100 may include a finger flange channel 140. The finger flange channel 140 is adapted to accept the finger flanges 116 of a pre-filled syringe 105 to effectively close around said finger flanges 116 in a close-fitting manner. As may be seen more closely in FIGS. 1 and 3, the finger flange channel 140 comprises a plurality of openings to allow for insertion of the finger flange 116 and is intended to be complementary to the size of the finger flange 116 to promote a close fit. The finger flange channel 140 is attached cooperatively in relation to the distal end 130 with the openings facing a direction of insertion of the syringe 105. The finger flange channel 140 may comprise an upper and lower portion with openings so defined as to fit the width of a plunger shaft 115plunger shaft 115 and opened at a vertical distance such that the distance between the vertical openings matches that of the height of the finger flange 116.

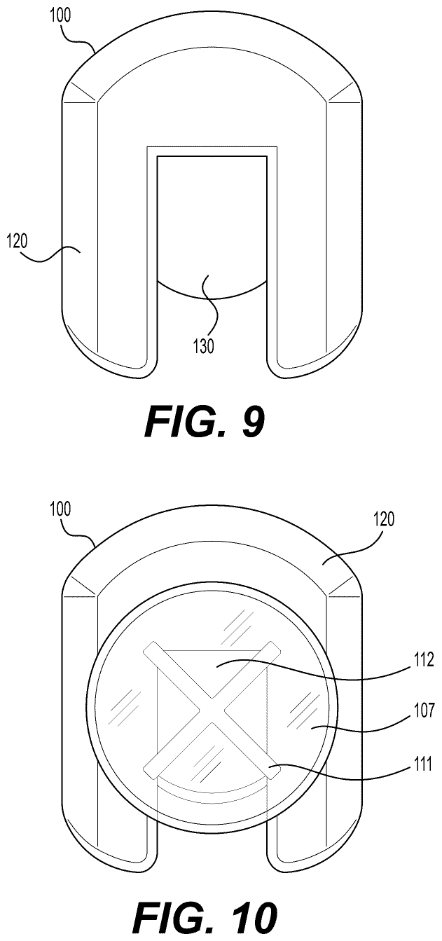

As seen more clearly in FIG. 9, the proximal end 120 of the tamper evident assembly 100, specifically the area extending above the finger flange channel 140, may extend horizontally or inwardly towards the plunger shaft 115plunger shaft 115. As shown in FIG. 10, when assembled with a pre-filled syringe 105, the proximal end 120 may extend horizontally or inwardly such that it encapsulates the widest part of the plunger shaft 115, specifically the plunger shaft protrusions 111, which assists by mechanically preventing rotation of the plunger shaft 115. The proximal end 120 additionally by virtue of its width and positioning when assembled in connection with the tip or thumb depression of the plunger rod 107 may block access to the plunger rod channels 112 where an unauthorized user may attempt to access the liquid of the pre-filled syringe 105 via the vulnerable plunger 108 as the plunger shaft channel 112 may allow space for items or tools such as an additional needle, paperclip, screwdriver, or other instrument to access the interior of the syringe body 106 via the plunger 108. Essentially, the proximal end 120 may assume a variety of different configurations and operative capabilities, including at least to prevent rotation of the plunger rod 107 and access to the plunger 108 via plunger rod channels 112.

In some preferred embodiments, the elongated shaft 110 is sized to allow for a close-fitting arrangement of the proximal and distal ends 120, 130 along the finger flange 116 and tip cover 109 of the pre-filled syringe 105. This additionally serves to prevent removal of the plunger rod 107 from the syringe 105.

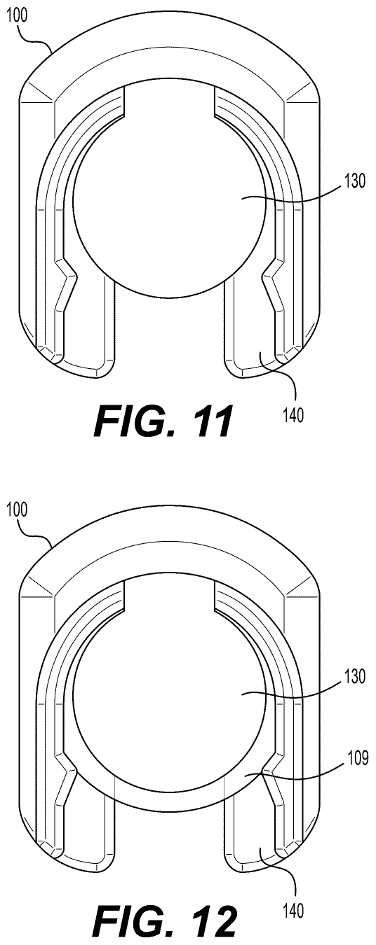

Additionally, the proximal end 120 may be configured or structured to include any one of a plurality of different mechanical enclosures to prevent tampering, as may be seen in FIGS. 11-13. The finger flange channel 140 may include additional protrusions to “lock” the plunger shaft channel 112 into place, the protrusions, and the finger flange channel 140 working in cooperation to prevent rotation of the plunger rod 107 thereby maintaining a “locked” position.

In certain embodiments, the elongated shaft 110 may be solid in nature or may have tapered points to create a hinge. The tapered points may occur closest to the proximal 120 or distal 130 end of the elongated shaft 110 and allow for either or both ends to easily bend, allowing for quick breakaway of the device when in use. The elongated shaft 110 may be formed from a less rigid, or semi rigid material where, when assembled, the proximal end 120 in connection with the finger flange 116 and the distal end 130 in connection with the tip cover 109 remains rigid and overlapping.

The pre-filled syringe 105 may be any standard sized pre-filled syringe, such as a 1 mL short syringe, or may be of various sizes, such as a 2.25 mL or 5 ml syringe. The tamper evident assembly 100 is generally used in a single-use delivery format.

The tamper evident assembly 100 may be comprised of any partially flexible and/or semi-rigid material, such as plastic, or potentially of any other material which would have a sufficient structural integrity to prevent loose orientation while maintaining the ability to be removed without excess tooling, preferably by hand. For example, the assembly 100 may be made of injection molded polypropylene.

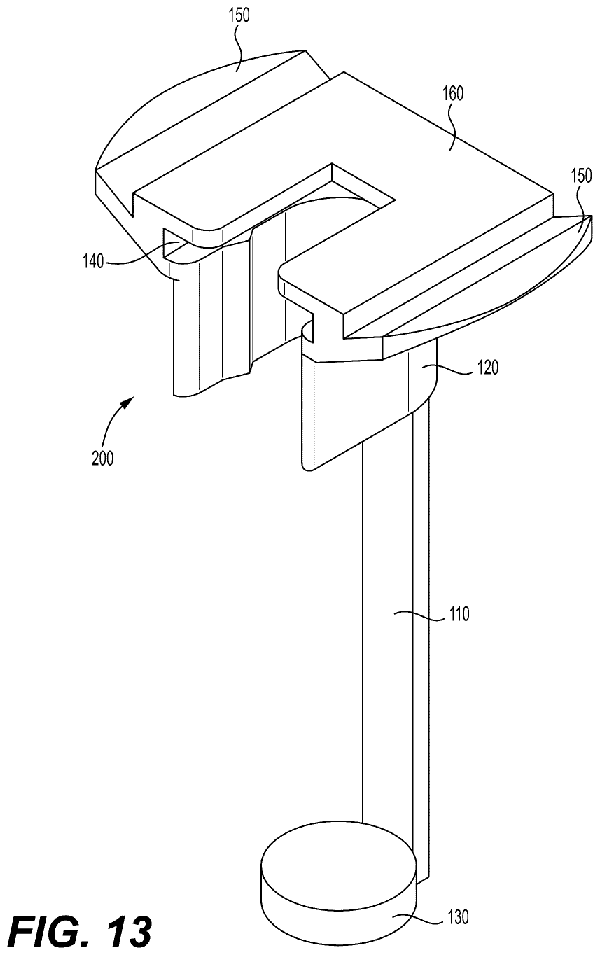

In yet another embodiment represented in FIG. 13, an assembly includes many of the operative features of the embodiments of FIGS. 1-12. This additional embodiment is directed towards a tamper evident or resistant assembly 200 to have additional grips 150 along the proximal end 120, along with a widened upper portion 160 of the proximal end 120. The grips 150 extend horizontally and from the finger flange channel 140 at the proximal end 120. The grips 150 may additionally be feathered in sizing or extend upwards at an angle which is not perpendicular from the proximal end 120. When operatively attached and positioned, the assembly 200 allows for better grip from the user while maintaining a small spatial footprint in medical storage.

In other embodiments, the tamper evident assembly 100 when assembled to the pre-filled syringe 105 may additionally include a film encompassing the entire assembly. The film may be PVC or polyolefin film which may be shrink wrapped or otherwise tightly fitted around the entire assembly. The film serves to provide an additional layer of evidence for tampering. Specifically, the anti-rotation aspects of the proximal end 120 interacting with the plunger shaft protrusions 111 in combination with the film serves as an added layer of protection for anti-tampering and provides tamper evidence. The anti-rotation aspect of the assembly 100 may prevent loosening or otherwise stretching of the film which could allow access for tampering. Additionally, the film surrounding the nearest area of the plunger shaft 115 would provide evidence of tampering by showing stretching or other distortion of the film absent the anti-rotation aspect of the assembly 100.

In additional embodiments, the film may additionally contain perforations along the length of the elongated shaft 110 to provide for ease of removal during intended use.

Also disclosed herein is a method of protecting a syringe containing a material, the method generally comprising receiving a pre-filled syringe, attaching to the pre-filled syringe a tamper resistant assembly, and enclosing the pre-filled syringe and tamper resistant assembly in a wrapping, where removal or other impairment of the wrapping indicates tampering. The tamper resistant assembly serves to mechanically block openings of the pre-filled syringe and to prevent rotation or depression of a plunger rod of the pre-filled syringe.

The distal end of the syringe body may take the form of a tip which may include a tip cap, such as a luer lock connector.

In some embodiments, the pre-filled syringe of the method may have a syringe body with a tip extending from a distal body end, opposite an open proximal body end. The open proximal body end allows access to a cavity in the syringe body, formed by the cylindrical shape of said syringe body, which may accept a liquid or semi-liquid material along with a plunger and plunger rod.

In some embodiments, the assembly has an elongated shaft with two protruding ends at the proximal and distal ends of said shaft. The proximal and distal protruding ends are configured to cover at least partially or fully the complimentary proximal and distal ends of the pre-filled syringe.

In some embodiments, the wrapping of the pre-filled syringe and assembly includes shrink-wrapped film.

In other embodiments, the shrink-wrapped film may additionally have perforations running along at least one length of the tamper resistant assembly as attached to the pre-filled syringe. The perforations may run in parallel to the length and direction of the elongated shaft of the assembly.

Since modifications, variations and changes in detail may be made to the described preferred embodiments of the invention, it is intended that all matters in the foregoing description and shown in the accompanying drawings be interpreted as not limited and illustrative.

The invention claimed is

1. A tamper resistant and/or tamper evident assembly for a syringe, said assembly comprising:
an elongated shaft;
a protruding proximal end and distal end, said proximal and distal ends configured to cover complimentary proximal and distal ends of a syringe; and,
a means of wrapping the syringe wherein removal of the wrapping indicates tampering;
wherein the assembly is configured to mechanically block openings of the syringe and prevent rotation or depression of a plunger rod of said syringe.

2. The assembly of claim 1, wherein the proximal end of the elongated shaft is configured to surround a finger flange of the syringe.

3. The assembly of claim 1, wherein the distal end of the elongated shaft is configured to block a tip or cap of the syringe.

4. The assembly of claim 3, wherein the tip or cap of the syringe is a luer-lock device.

5. The assembly of claim 1, wherein the means of wrapping the syringe comprises shrink wrap.

6. The assembly of claim 5, wherein the shrink wrap has perforations running parallel to the length of the elongated body.

7. The assembly of claim 5, wherein the shrink wrap is comprised of an opaque or clear material, such as plastic.

8. The assembly of claim 1, wherein the assembly is comprised of a lightweight, disposable material such as polypropylene.

9. The assembly of claim 2, wherein the proximal end of the elongated body surrounding the finger flange of the syringe is additionally configured to fit in a complementary manner to the construction of the plunger rod of the syringe via additional projections into depressions of the plunger rod.

10. The assembly of claim 1, wherein the syringe is a pre-filled syringe.

11. The assembly of claim 1, wherein the elongated shaft additionally comprises a hinge adjacent to the protrusions of the distal or proximal end.

12. A method of protecting a syringe containing a material, the method comprising:
receiving a pre-filled syringe having a syringe body with a tip extending from a distal body end and an open proximal body end, wherein the open proximal body end allows for a plunger and plunger rod to be inserted into the syringe body, and wherein the syringe body is filled with a material;
attaching to the pre-filled syringe a tamper resistant assembly comprising an elongated shaft, a protruding proximal end and distal end, said proximal and distal ends configured to cover complimentary proximal and distal ends of the pre-filled syringe; and, a means of wrapping the pre-filled syringe as attached to the assembly wherein removal of the wrapping indicates tampering;
wherein the assembly is configured to mechanically block openings of the pre-filled syringe and prevent rotation or depression of a plunger rod of said pre-filled syringe.

13. The method of claim 12, wherein the material comprises medication in a liquid form.

14. The method of claim 12, wherein the tip of the syringe body may additionally include a tip cap, such as a luer lock connection.

15. The method of claim 12, wherein the means of wrapping the pre-filled syringe includes attaching a shrink-wrapped film over the tamper resistant assembly as attached to the pre-filled syringe.

16. The method of claim 12, wherein attaching the film includes attaching the film to the syringe.

17. The method of claim 15, wherein the shrink-wrapped film may additionally comprise perforations along at least one length of the tamper resistant assembly as attached to the pre-filled syringe.
